(19) United States
(12) Patent Application Publication
(10) Pub. No.: US 2004/0161469 A1
(43) Pub. Date: Aug. 19, 2004

(54) Method for producing sterile active pharmaceutical substances

(76) Inventor: Klaus-Peter Volkl, Magdeburg (DE)

Correspondence Address: WILLIAM COLLARD, COLLARD & ROE, P.C., 1077 NORTHERN BOULEVARD, ROSLYN, NY, 11576, US

(21) Appl. No.: 10/778,814

(22) Filed: Feb. 13, 2004

(30) Foreign Application Priority Data
Feb. 17, 2003 (DE) 103 06 761.2

(51) Int. Cl. A61K 9/14

(52) U.S. Cl. 424/489

(57) ABSTRACT

An improved method for producing sterile active pharmaceutical substances is provided, which achieves an increased throughput per time unit with a reduced expenditure of time and energy, as well as a reduced use of solvent, by means of a combination of washing and sterile filtration.

### CROSS REFERENCE TO RELATED APPLICATIONS

Applicant claims priority under 35 U.S.C. §119 of German Application No. 103 06 761.2 filed Feb. 12, 2003.

1. Field of the Invention

The present invention relates to a method for producing sterile active pharmaceutical substances such as cytostatics, steroids, antibiotics, or others, whereby the active substances to be sterilized are first introduced into a solvent in powder form, the solution that forms is subsequently subjected to sterile filtration, and the sterile active substance (cytostatic, steroid, antibiotic, or other) is then separated from the solvent or dried.

2. The Prior Art

A number of pharmaceutically utilized substances, including cytostatics, steroids, and antibiotics, must be present in sterile form, i.e. free of any contaminants, including those of microbial origin.

Methods for the production and purification of an antibiotic, such as ampicillin, for example, have been known for a long time (DE 16 45 980 A1, DE 21 42 180 A1, DE 32 08 506 A1).

DE 16 45 980 A1 proposes dissolving crude ampicillin in a suitable water-miscible solvent, with the addition of a dissolution agent, such as an acid, filtering this solution in order to obtain a completely clear solution, then precipitating the ampicillin by adding a suitable base, finally filtering off the resulting precipitate and washing it in several process steps and finally drying the pressed filter cake. Due to the fact that degrees of saturation of only a maximum of 20% can be achieved, it is easily understandable for a person skilled in the art that this method is characterized by significant costs, resulting from an elevated consumption of solvent and an elevated expenditure of time.

It is true that the degree of saturation can be increased, within limits, by means of conducting the temperature of the process in a certain way, as disclosed in greater detail in the documents DE 21 42 180 A1 and DE 32 08 506 A1, but this process results in additional expenditures that are particularly represented by an elevated consumption of energy.

### SUMMARY OF THE INVENTION

Proceeding from this starting point, it is an object of the present invention to provide an improved method for producing sterile active pharmaceutical substances such as cytostatics, steroids, antibiotics, or others, with which an increased throughput can be achieved as compared with the state of the art described initially, at a lower expenditure of time and energy, as well as a lower use of solvent.

This object is achieved, according to the invention, by a method for producing sterile active pharmaceutical substances such as cytostatics, steroids, antibiotics, or others, according to which the active substance, in each instance, is first introduced into a solvent in powder form, subsequently the solution that forms is subjected to sterile filtration and then the sterile active substance is separated from the solvent or dried. According to the invention, the particles of the active substance are washed in the solvent, i.e. freed of contaminants, and sterilized, so that the outer layer of the particles with all the contaminants, including those of microbial origin, peels off and is taken up by the solvent and transported away. Undissolved but purified particle portions settle as sediment. The solvent containing the contaminants and dissolved particle portions is passed to sterile filtration. Subsequently, the sediment of purified, sterile particle portions that was obtained is combined with the sterilized filtrate, and this combination is finally separated into solvent and sterile active substance, in a reaction vessel.

According to a particularly preferred embodiment, the solvent flows through the particles of the active substance during the washing process, counter to gravitation and/or counter to a centrifugal force.

The purified, sterile particle portions of the sediment, plus the sterilized filtrate, may be dried by means of a temperature change. Both aqueous and organic solvents can be used in the process. The solvent that has been separated from the sediment and the sterile filtrate by means of drying may also be passed back to the process.

In another aspect, a medication is provided that contains sterile active pharmaceutical substances, such as cytostatics, steroids, antibiotics, etc., which in turn are produced according to the method according to the invention.

The method according to the invention has the significant advantage, as compared with conventional methods, that despite relatively low degrees of saturation in the solvent, of only 20 g/L Δ2%, in part, a higher throughput per time unit is recorded. Complete dissolution of the active substance in the solvent used for sterilization of the same is unnecessary. After extensive tests, it was found sufficient to merely loosen the outer layer of the particles by means of washing. As the contaminants adhere to the outer layer of the particles, they can be taken up and transported away by the solvent.

### BRIEF DESCRIPTION OF THE DRAWINGS

Other objects and features of the present invention will become apparent from the following detailed description considered in connection with the accompanying drawing. It should be understood, however, that the drawing is designed for the purpose of illustration only and not as a definition of the limits of the invention.

In the drawing,

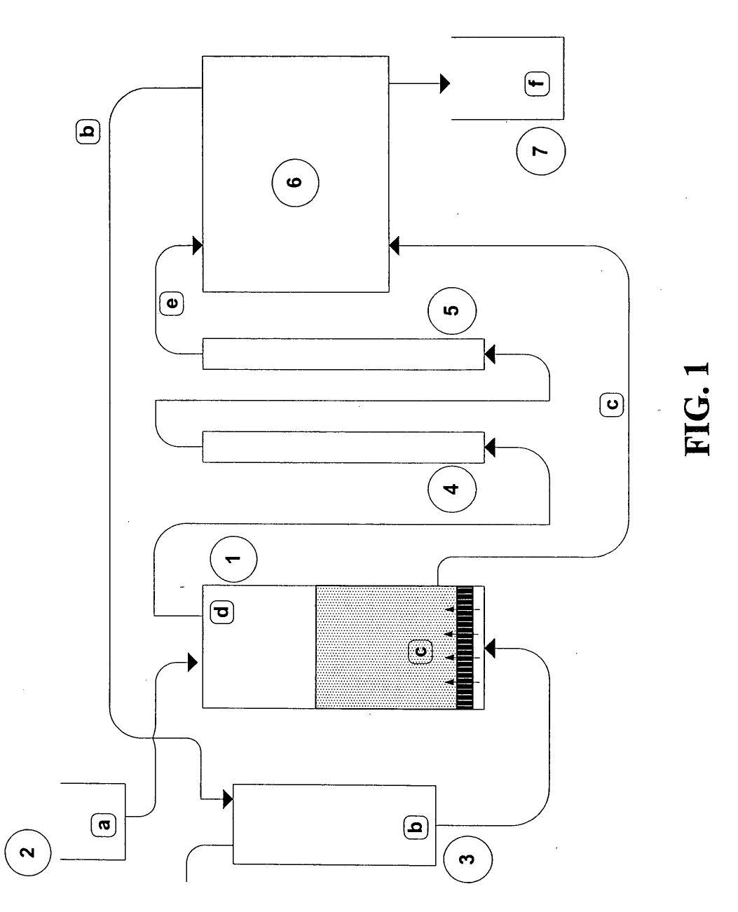

The single figure, FIG. 1, schematically shows a flow diagram illustrating an embodiment of the invention.

### DETAILED DESCRIPTION OF PREFERRED EMBODIMENTS

According to the flow chart of FIG. 1, a suspension of an active pharmaceutical substance a (non-sterile) made available in powder form (crystalline and/or amorphous state) and a solvent b is first produced in a reaction vessel “washing” 1. Both aqueous and organic solvents b, which are known, in and of themselves, can be used as solvent b. Active substance a and the solvent b are passed to reaction vessel 1 from a supply vessel 2 and 3, respectively.

Subsequently, the particles of active substance a are washed in reaction vessel 1, i.e. freed of contaminants and sterilized, so that an outer layer of the particles plus these contaminants, which can also be of microbial origin, peels off and is taken up by solvent b.

As the result of the experiments already mentioned above, it has been proven to be particularly advantageous to have solvent b flow through active substance a during the washing process, counter to gravitation and/or counter to a centrifugal force, not shown in greater detail.

The duration of the above washing process is selected as a function of the amount of active substance a used, and a suitable volume of solvent b.

After discontinuation of the washing process, i.e. the flow through the particles, undissolved but purified (sterile) particle portions of active substance a settle on the bottom of reaction vessel 1, or on a filter plate of reaction vessel 1, not designated specifically here, as sediment c.

The solvent d, which contains the contaminants and dissolved particle portions, on the other hand, is passed to sterile filtration, by means of a pump that is known, in and of itself, and is not shown in greater detail here; the sterile filtration includes a 5 μm prefilter 4 and a subsequent 0.2 μm filter 5 suitable for sterile filtration.

Subsequent to this filtration, the sterilized filtrate e can be combined directly with the particle portions that are present in reaction vessel 1 as sediment c, and this combination can be passed to a reaction vessel “separation” 6 of the particle portions, whereby the separation of the sterile active substance f from used solvent b takes place, preferably by means of drying.

Drying of the resulting combination of sterile sediment c and sterilized filtrate e can be carried out by means of a temperature change, as is known, i.e. by means of heat or freeze-drying, in reaction vessel “separation” 6. Separated solvent b can furthermore be passed back to the process.

In a final step, sterile active pharmaceutical substance f is passed to a suitable supply vessel 7.

Accordingly, although only at least one embodiment of the present invention have been shown and described, it is to be understood that many changes and modifications may be made thereunto without departing from the spirit and scope of the invention as defined in the appended claims.

What is claimed is:

1. A method for producing a sterile active pharmaceutical substance comprising the steps of:
(a) introducing an active substance in powder form into a solvent;
(b) washing and sterilizing particles of the active substance in the solvent so that an outer layer of the particles with contaminants peels off and is taken up by the solvent and undissolved but purified particle portions settle as sediment;
(c) subjecting the solvent containing the contaminants and dissolved particle portions to sterile filtration to obtain a sterilized filtrate;
(d) subsequently combining the sterilized filtrate with the sediment of purified particle portions; and
(e) separating the sterile active substance from the solvent to produce a sterile active pharmaceutical substance.

2. The method according to claim 1, wherein during washing the solvent flows through the particles of the active substance counter to gravitation or counter to a centrifugal force.

3. The method according to claim 1, wherein the sterile active substance is separated from the solvent by subjecting the sterilized filtrate and sediment following combination to a temperature change to dry off the solvent.

4. The method according to claim 1, wherein the solvent is an aqueous solvent or an organic solvent.

5. The method according to claim 3, wherein the solvent separated from the sediment plus the sterilized filtrate by means of drying is passed back for reuse in the method.

6. A medication containing a sterile active pharmaceutical substance produced by the method according to claim 1.
